(19) United States
(12) Patent Application Publication
(10) Pub. No.: US 2008/0004655 A1
(43) Pub. Date: Jan. 3, 2008

(54) Automatic lock mechanism for bar clamp

(75) Inventor: Johnny J. T. Chiu, Hsichih City (TW)

Correspondence Address: BRUCE H. TROXELL, SUITE 1404, 5205 LEESBURG PIKE, FALLS CHURCH, VA, 22041, US

(73) Assignee: Valentine International Ltd.

(21) Appl. No.: 11/478,636

(22) Filed: Jul. 3, 2006

(51) Int. Cl. A61B 17/00

(52) U.S. Cl. 606/205

(57) ABSTRACT

A bar clamp includes a forward gripping section having two jaws, a rear housing, and an interconnecting tube. Pressing back a trigger handle will cause the jaws to grip an article, and counterclockwise pivot a link to cause a lock member to move through an opening of a locking plate into a slot of a release member. The article is gripped permanently after releasing the trigger handle by lockingly engaging a top of the lock member with an upper face of the opening. Counterclockwise pivoting a release lever will disengage the lock member with the opening, release a stored elastic force and apply same to the trigger handle for clockwise pivoting, and move one jaw away from the other jaw for dropping the article.

### BACKGROUND OF THE INVENTION

1. Field of Invention

The present invention relates to bar clamps and more particularly to a bar clamp having an improved lock mechanism, the bar clamp being adapted to operate by a single hand for gripping an article (e.g., one being difficult of accessing or unwilling to access by the hand(s)) and automatically locking same even after the hand releasing the gripped article.

2. Related Art

In many contexts (e.g., in hospitals), persons (e.g., medical workers) may have difficulties of accessing an article or are unwilling to access same by the hand(s) due to potential contamination. Thus, a person may use a clamp (e.g., bar clamp) to grip the article.

Typically, a bar clamp has an elongate bar, two front jaws, and a rear pistol-shaped lock section including a handgrip and a trigger handle. In use, one hand grips the handgrip and the trigger handle and presses back the trigger handle to activate the jaws in order to grip an article. Next, slide a release level to a locked position so as to continuously grip the article. Finally, slide the release level to an unlocked position for releasing the article for subsequent treatment.

However, the prior art bar clamp suffered from a disadvantage. For example, a person is prone to loosening the gripping by the hand after gripping the article by the jaws. Unfortunately, the gripped article may drop if it has not been locked (i.e., the bar clamp does not lock the gripped article automatically). In brief, the prior art bar clamp contradicts the habit of using same by ordinary people. Thus, a need for improvement exists in order to overcome the inadequacy of the prior art and contribute significantly to the advancement of the art.

### SUMMARY OF THE INVENTION

It is therefore an object of the present invention to provide a bar clamp having a unique and novel lock mechanism which is operable to lock an article gripped by jaws automatically even after the hand gripping the article is loosened. For releasing the article, the user may slide a release lever to an unlocked position.

To achieve the above and other objects, the present invention provides a bar clamp comprising a gripping section including two pairs of first arm and second arm formed of an elastomeric material, and two jaws each formed at one ends of either pair of first arm and second arm; a tube for receiving portions of the first arms and including a forward chuck secured to the other ends of the first arms; and a housing including a forward channel with a rear portion of the tube secured therein, a curved groove, a handgrip, a pivotal trigger handle, a pivotal release member including two laterally projecting release levers, a hooked base, and an intermediate slot between the release levers, a connecting member in the rear portion of the tube and having one end secured to the other ends of the first arms and the other end projected rearward to secure to an intermediate portion of the trigger handle, a rectangular locking plate having a top retained in the groove and a bottom retained in the base, the locking plate including an intermediate opening, and a link having one end secured to the intermediate portion of the trigger handle and including a rear curved lock member; whereby pressing back the trigger handle will counterclockwise pivot the trigger handle to pull the connecting member and the second arms with an elastic force stored in the second arms, move pairs of first arm and second arm toward each other until an article is gripped by the jaws, and counterclockwise pivot the link to cause the lock member to move through the opening into the slot wherein the article is gripped permanently after releasing the trigger handle by lockingly engaging a top of the lock member with an upper face of the opening; and counterclockwise pivoting the release lever will counterclockwise pivot a bottom of the locking plate about the groove, disengage the top of the lock member with the upper face of the opening, release the elastic force and apply same to the trigger handle to clockwise pivot both the link and the trigger handle, and push the connecting member and the pairs of second arm to move one pair of first arm and second arm away from the other pair of first arm and second arm for dropping the article.

In one aspect of the present invention the lock member is a bifurcation including two branches, and the opening comprises two holes corresponding to the branches for either allowing the branches to freely pass or lockingly retaining same therein.

In another aspect of the present invention a top of either branch has an uneven surface.

The above and other objects, features and advantages of the present invention will become apparent from the following detailed description taken with the accompanying drawings.

### BRIEF DESCRIPTION OF THE DRAWINGS

FIG. 1 is a side elevation of a preferred embodiment of bar clamp according to the invention;

FIG. 2 is a longitudinal sectional view of the rear lock section;

FIG. 3 is a longitudinal sectional view of the housing of the lock section;

FIG. 4 is a longitudinal sectional view of the trigger handle;

FIG. 5 is a perspective view of the sliding member;

FIG. 6 is a side elevation of the link;

FIG. 7 is a perspective view of the locking plate;

FIG. 8 is a perspective view of the release member;

FIG. 9 is a longitudinal sectional view of the locking plate;

FIG. 10 schematically depicts movement of the locking plate in the lock section in an unlocking operation; and

FIG. 11 is a longitudinal sectional view of the locking plate locked by the curved members of the link.

### DETAILED DESCRIPTION OF THE INVENTION

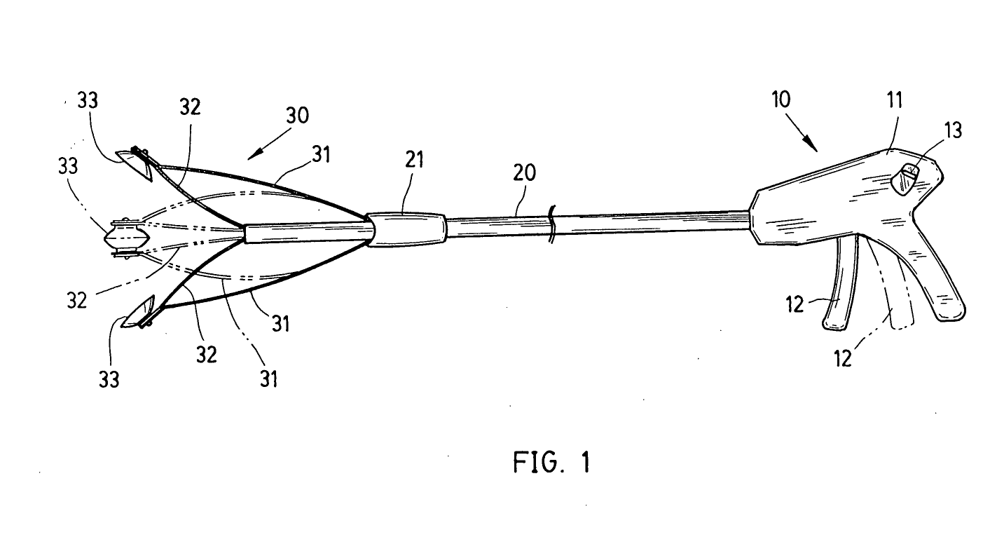

Referring to FIG. 1, a bar clamp in accordance with a preferred embodiment of the invention comprises a rear lock section 10, an intermediate tube 20, and a front gripping section 30. Each component is discussed in detailed below.

The lock section 10 comprises a housing 11 of pistol shape, a trigger handle 12, and a release member 13. The tube 20 comprises a forward chuck 21. The gripping section 30 comprises two pairs of first arm 31 and second arm 32, and two cup-shaped jaws 33 each formed at open ends of either pair of first arm 31 and second arm 32.

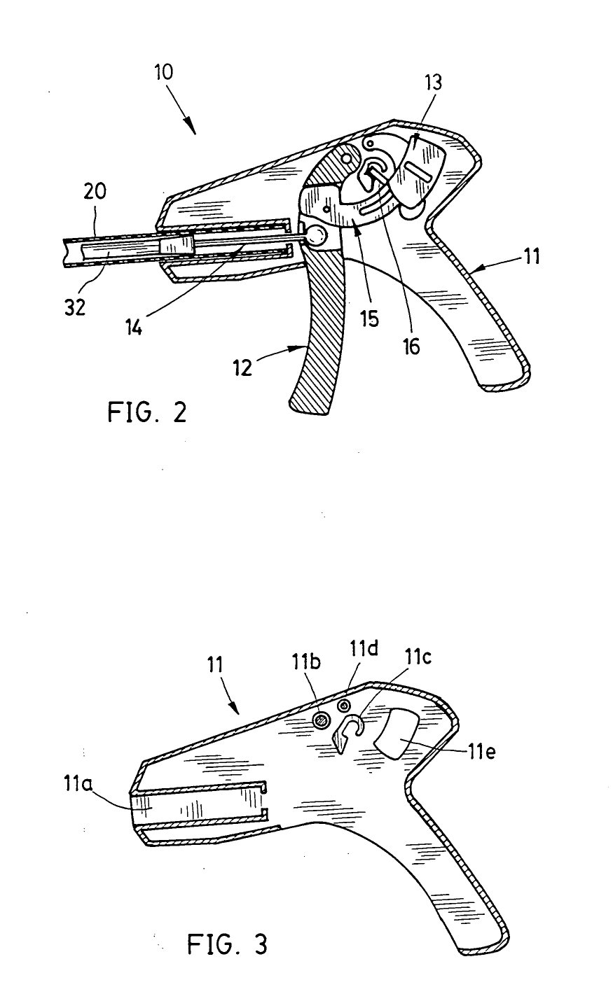

The chuck 21 is adapted to fasten the proximal ends of the first arms 31. The other ends of the second arms 32 are secured to one end of a connecting member 14 (see FIG. 2) in a rear of the tube 20. The other end of the connecting member 14 is secured to the trigger handle 12 as detailed later.

Each of the first and second arms 31 and 32 is a flexible strip formed of an elastomeric material. The first arms 31 are adapted to bend when portions of the second arms 32 are pulled into the tube 20. As a result, the two pairs of first arm 31 and second arm 32 move toward each other with an elastic force being stored. The elastic force may pull the pairs of first arm 31 and second arm 32 apart automatically when the pulling is removed.

In operation, one hand may press back the trigger handle 12 to pull the second arms 32 further into the tube 20 by moving the pairs of first arm 31 and second arm 32 toward each other until an article (not shown) is gripped by the jaws 33.

Note that the jaw 33 may be shaped other than cup in other embodiments without departing from the scope and spirit of the invention as long as a secure gripping of an article is made possible by the jaws 33. Further note that the tube 20, the chuck 21, the first arms 32, and the jaws 33 are well known devices.

Referring to FIG. 2, the lock section 10 further comprises a connecting member 14, a link 15, and a locking plate 16. The connecting member 14 is provided in the rear of the elongate tube 20 in the lock section 10. In detail, the elongate connecting member 14 has a rear end secured to the trigger handle 12 and a front end secured to the second arms 32 which have portions extended from the tube 20 to secure to the jaws 33.

Referring to FIG. 3, the pistol-shaped housing 11 consists of two mated halves and comprises a forward channel 11a, a first pivot pin 11b, a curved groove 11c, a second pivot pin 11d, and an opening 11e open to both sides of the housing 11. The channel 11a is adapted to retain the rear of the tube 20. The first pivot pin 11b is pivotably connected to an upper end of the trigger handle 12 such that the trigger handle 12 is able to pivot a predetermined angle about the first pivot pin 11b. The groove 11c is adapted to retain an upper part of the locking plate 16 such that the locking plate 16 is able to pivot a predetermined angle about the groove 11c. The second pivot pin 11d is pivotably connected to the release member 13 such that the release member 13 is able to pivot a predetermined angle about the second pivot pin 11d. The opening 11e is adapted to allow parts of the release member 13 to project from the housing 11 for facilitate operation. Note that both the first and second pivots 11b and 11d pivotably connected to holes of the housing 11 are well known in the art and a detailed description thereof is therefore deemed unnecessary.

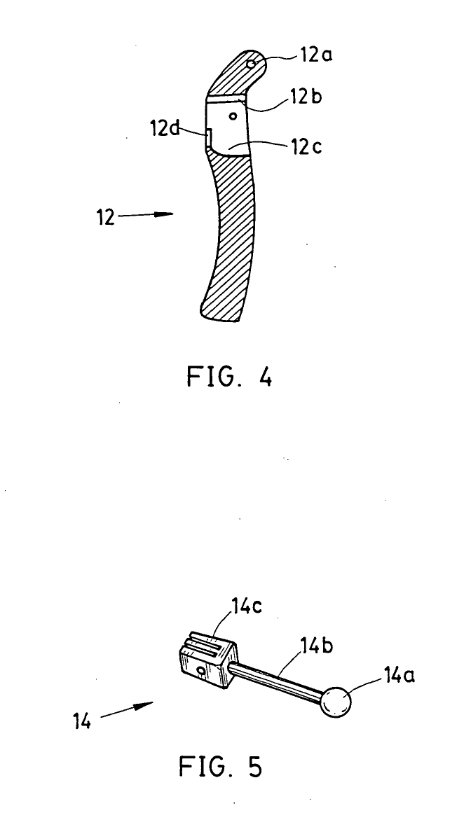

Referring to FIGS. 4 and 5 in conjunction with FIG. 2, the trigger handle 12 a slightly curved member and comprises an upper first aperture 12a, a passage 12c below the first aperture 12a, a well 12b in an upper portion of the passage 12c, and a cavity 12d adjacent the passage 12c and being in communication with the passage 12c through an aperture. The first pivot pin 11b is inserted into the first aperture 12a so as to be pivotal about the housing 11. The link 15 has a forward end secured to the well 12b in the passage 12c. The passage 12c is adapted to receive an enlarged head 14a of the connecting member 14. The cavity 12d is adapted to receive a portion of a linking bar 14b of the connecting member 14. The head 14a is prohibited from entering into the cavity 11d because the aperture interconnecting the cavity 11d and the passage 12c is smaller than the head 14a.

Referring to FIG. 5 specifically, in addition to the rear head 14a and the intermediate linking bar 14b the connecting member 14 further comprises a forward connection member 14c. The connection member 14c and most portion of the linking bar 14 are retained in the tube 20 (i.e., in the channel 11a). The connection member 14c is secured to the other ends of the second arms 32. In operation, pressing back the trigger handle 12 will pull both the connecting member 14 and the second arms 32 rearward.

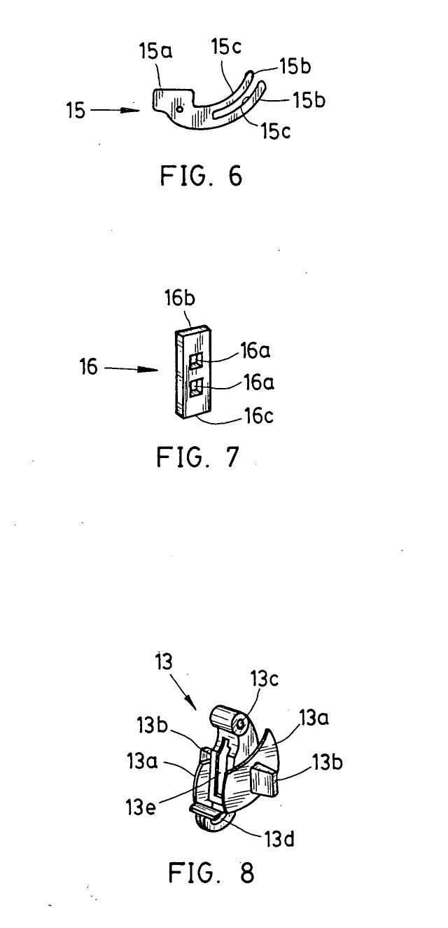

Referring to FIG. 6, the slightly curved link 15 comprises a forward member 15a secured to the well 12b of the trigger handle 12, and two rear curved members 15b each having an uneven upper face 15c for the sake of increasing friction as detailed later.

Referring to FIG. 7, the rectangular locking plate 16 comprises a top 16b, a bottom 16c, and two intermediate holes 16a spaced apart by a predetermined distance. The holes 16a are large enough to either allow the curved members 15b to freely pass or allow same to lockingly retain therein. The locking plate 16 has a predetermined thickness. The top 16b is retained in the groove 11c and the bottom 16c is retained in the release member 13 as detailed later.

Referring to FIG. 8, the release member 13 comprises two side plates 13a each having a laterally projecting release lever 13b, a top hollow cylinder 13c, a hooked base 13d, and a slot 13e between the plates 13a. The plates 13a are mounted on inner surfaces of the housing 11 at both mouths of the opening 11e. The second pivot pin 11d has a diameter slightly smaller than the bore of the cylinder 13c. Thus, the second pivot pin 11d is adapted to insert through the cylinder 13c to pivotably connect the release member 13 in the housing 11. The base 13d is adapted to retain the bottom 16c of the locking plate 16 and is adapted to allow same to pivot a predetermined angle. Thus, the locking plate 16 may change an angle relative to the housing 11 as the release member 13 pivots. The slot 13e is adapted to allow the curved members 15b to pass freely without interfering with the release member 13 in operation.

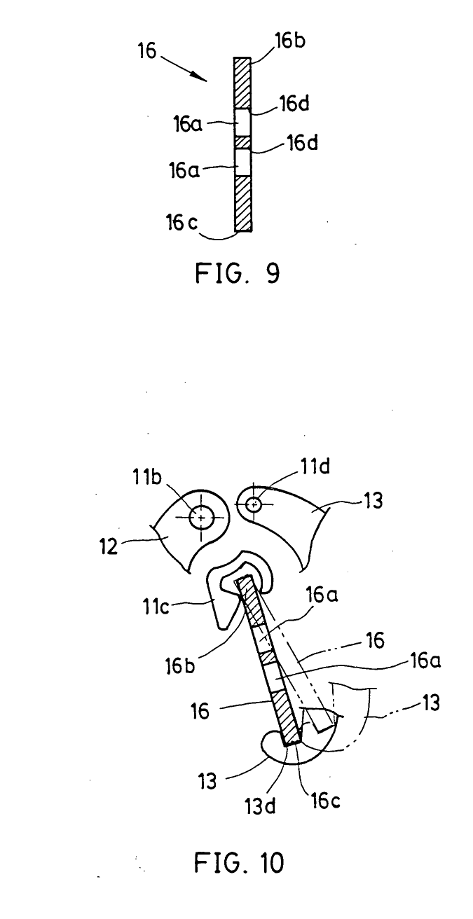

Referring to FIG. 9, the locking plate 16 further comprises an upper face 16d in each hole 16a. Each of the curved members 15b is adapted to lockingly contact the upper face 16d.

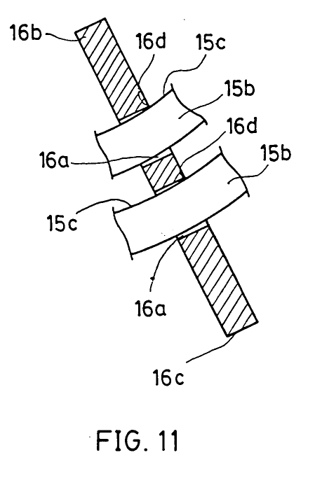

Referring to FIGS. 10 and 11 in conjunction with FIGS. 1 to 9, the top 16b of the locking plate 16 is retained in the groove 11c and the bottom 16c thereof is retained in the base 13d. As stated above, the release member 13 is pivotal about the second pivot pin 11d. An article gripping and locking operation of the invention will be described in detailed below. A user may use one hand to grip both the trigger handle 12 and a fixed handgrip of the housing 11 and press the trigger handle 12 backward. And in turn, the trigger handle 12 pivots counterclockwise about the first pivot pin 11b. As such, the connecting member 14 and the second arms 32 are pulled rearward. Also, the pairs of first arm 31 and second arm 32 move toward each other until an article is gripped by the jaws 33. At the same time, the link 15 secured to the trigger handle 12 pivots counterclockwise about the first pivot pin 11b. And in turn, the curved members 15b freely move through the holes 16b of the stationary locking plate 16 into the slot 13e. Once the article is gripped by the jaws 33, the user may stop gripping the trigger handle 12 or even remove the hand from the trigger handle 12. A forward force (i.e., the stored elastic force of the second arms 32) is immediately applied to the trigger handle 12 to attempt to push the connecting member 14 and the pairs of second arm 32 forward. However, the trigger handle 12, the link 15, and the locking plate 16 remain stationary (i.e., locked) as detailed below. A potential clockwise pivotal movement of the curved members 15b is prohibited since the engagements of the upper faces 15c of the curved members 15b with the upper faces 16d in the holes 16a of locking plate 16 are lockingly tight due to the uneven upper faces 15c (see FIG. 11). By configuring as above, once an article is gripped a user does not have to worry about the article being dropped onto the ground after releasing the trigger handle since the article is locked automatically.

An unlocking operation (i.e., releasing the gripped article) of the invention will be described in detailed below. A user may pivot the release lever 13 counterclockwise (i.e., the release lever 13 counterclockwise pivots about the second pivot pin 11d). And in turn, the bottom 16c of the locking plate 16 pivots counterclockwise about the groove 11c (see dash lines of FIG. 10). As such, the upper faces 15c of the curved members 15b are disengaged with the upper faces 16d of the holes 16a in the locking plate 16. Once the curved members 15b are free, the forward force (i.e., the stored elastic force of the second arms 32) is immediately applied to the trigger handle 12 and clockwise pivot both the link 15 and the trigger handle 12 in order to push the connecting member 14 and the pairs of second arm 32 forward. As such, one pair of first arm 31 and second arm 32 move away from the other pair of first arm 31 and second arm 32. As a result, the article is no longer gripped.

While the invention herein disclosed has been described by means of specific embodiments, numerous modifications and variations could be made thereto by those skilled in the art without departing from the scope and spirit of the invention set forth in the claims.

What is claimed is:

1. A bar clamp comprising:
a gripping section including two pairs of first arm and second arm formed of an elastomeric material, and two jaws each formed at one ends of either pair of first arm and second arm;
a tube for receiving portions of the first arms and including a forward chuck secured to the other ends of the first arms; and
a housing including a forward channel with a rear portion of the tube secured therein, a curved groove, a handgrip, a pivotal trigger handle, a pivotal release member including two laterally projecting release levers, a hooked base, and an intermediate slot between the release levers, a connecting member in the rear portion of the tube and having one end secured to the other ends of the first arms and the other end projected rearward to secure to an intermediate portion of the trigger handle, a rectangular locking plate having a top retained in the groove and a bottom retained in the base, the locking plate including an intermediate opening, and a link having one end secured to the intermediate portion of the trigger handle and including a rear curved lock member; whereby:
pressing back the trigger handle will counterclockwise pivot the trigger handle to pull the connecting member and the second arms with an elastic force stored in the second arms, move pairs of first arm and second arm toward each other until an article is gripped by the jaws, and counterclockwise pivot the link to cause the lock member to move through the opening into the slot wherein the article is gripped permanently after releasing the trigger handle by lockingly engaging a top of the lock member with an upper face of the opening; and
counterclockwise pivoting the release lever will counterclockwise pivot a bottom of the locking plate about the groove, disengage the top of the lock member with the upper face of the opening, release the elastic force and apply same to the trigger handle to clockwise pivot both the link and the trigger handle, and push the connecting member and the pairs of second arm to move one pair of first arm and second arm away from the other pair of first arm and second arm for dropping the article.

2. The bar clamp of claim 1, wherein the top of the lock member has an uneven surface.

3. The bar clamp of claim 1, wherein the lock member is a bifurcation including two branches.

4. The bar clamp of claim 3, wherein the opening comprises two holes corresponding to the branches for either allowing the branches to freely pass or lockingly retaining the branches therein.

5. The bar clamp of claim 3, wherein a top of either branch has an uneven surface.
